(19) United States
(12) Patent Application Publication
(10) Pub. No.: US 2016/0287286 A1
(43) Pub. Date: Oct. 6, 2016

(54) URETHRAL-INSERTION DEVICE

(71) Applicant: TERUMO MEDICAL INNOVATION, INC., Mountain View, CA (US)

(72) Inventors: Masakatsu KAWAURA, Sunnyvale, CA (US); Nao MANDAI, Mountain View, CA (US)

(73) Assignee: TERUMO MEDICAL INNOVATION, INC., Mountain View, CA (US)

(21) Appl. No.: 15/001,739

(22) Filed: Jan. 20, 2016

Related U.S. Application Data
(60) Provisional application No. 62/105,344, filed on Jan. 20, 2015.

(51) Int. Cl. A61B 17/34; A61B 17/04; A61F 2/00

(52) U.S. Cl. CPC A61B 17/3468; A61F 2/0045; A61B 17/0469; A61F 2002/0072; A61B 2017/306

(57) ABSTRACT

A urethral-insertion device configured to be inserted into a urethra is disclosed, which includes an elongated main body; a bladder suction port adapted to attract a bladder by suction, the bladder suction port located on the main body; and a urethra suction port adapted to attract a urethra wall by suction, the urethra suction port located on a distal side of the bladder suction port.

### CROSS-REFERENCES TO RELATED APPLICATIONS

This application contains subject matter disclosed in U.S. Application No. 62/105,344 filed on Jan. 20, 2015, which is incorporated herein by reference in its entirety.

### TECHNICAL FIELD

The present disclosure generally relates to a urethral-insertion device.

### BACKGROUND DISCUSSION

In a patient suffering from urinary incontinence, particularly stress urinary incontinence, urine leakage can occur due to an abdominal pressure exerted during a normal exercise or by laughing, coughing, sneezing or the like. This can be attributable, for example, to loosening of the pelvic floor muscle, which is a muscle for supporting the urethra, caused by childbirth or the like.

For treatment of urinary incontinence, surgical therapy can be effective, in which there is used, for example, a tape-shaped implant called “sling,” and the sling is placed indwelling in the body, so as to support the urethra thereby (see, for example, Japanese Patent Laid-Open No. 2010-99499). In order to put a sling indwelling in the body, an operator incises the vagina with a surgical knife, dissects a biological tissue between the urethra and the vagina, and forms a puncture-through hole for providing communication between the dissected biological tissue and the exterior, by use of a puncture needle. Then, the sling can be inserted into the puncture-through hole, to be placed to indwell in the dissected biological tissue in the body.

### SUMMARY

A urethral-insertion device is disclosed by which puncturing of a living body tissue by a puncture needle in the process of placing an embeddable implant (sling) for treatment of urinary incontinence indwelling into a living body can be performed relatively accurately and safely.

In accordance with an exemplary embodiment, a urethral-insertion device configured to be inserted into a urethra is disclosed, comprising: an elongated main body; a bladder suction port adapted to attract a bladder by suction, the bladder suction port being located on the main body; and a urethra suction port adapted to attract a urethra wall by suction, the urethra suction port being located on a distal side of the bladder suction port.

In accordance with an exemplary embodiment, a method is disclosed of placing a sling for use in treatment of urinary incontinence indwelling into a living body comprising: inserting a urethral-insertion device into a urethra; attracting by suction an inner wall of a bladder by a bladder suction port of the urethral-insertion device; moving a bladder from an original position of the bladder; and placing the sling indwelling into the living body between a vagina and the urethra.

### BRIEF DESCRIPTION OF THE DRAWINGS

FIG. 1 is a perspective view illustrating a first embodiment of a urethral-insertion device;

FIGS. 2A, 2B, 3A, and 3B are side views illustrating a procedure of operating the urethral-insertion device shown in FIG. 1;

FIG. 4 is a perspective view illustrating a second embodiment of the urethral-insertion device;

FIG. 5 is a perspective view illustrating a third embodiment of the urethral-insertion device;

FIG. 6 is a perspective view illustrating a fourth embodiment of the urethral-insertion device;

FIG. 7 is a perspective view illustrating a fifth embodiment of the urethral-insertion device;

FIGS. 8A and 8B are perspective views illustrating a sixth embodiment of the urethral-insertion device;

FIG. 9A is a perspective view illustrating a seventh embodiment of the urethral-insertion device;

FIG. 9B is a sectional view, taken along line 9B-9B of FIG. 9A, of the urethral-insertion device illustrated in FIG. 9A;

FIG. 10A is a perspective view illustrating an eighth embodiment of the urethral-insertion device;

FIG. 10B is a sectional view, taken along line 10B-10B of FIG. 10A, of the urethral-insertion device illustrated in FIG. 10A;

FIG. 11 is a perspective view illustrating a ninth embodiment of the urethral-insertion device;

FIGS. 12A and 12B are sectional views, taken along line 12-12 of FIG. 11, of the urethral-insertion device illustrated in FIG. 11;

FIG. 13A is a perspective view illustrating a tenth embodiment of the urethral-insertion device;

FIG. 13B is a sectional view, taken along line 13B-13B of FIG. 13A, of the urethral-insertion device illustrated in FIG. 13A;

FIG. 14A is a perspective view illustrating the tenth embodiment of the urethral-insertion device;

FIG. 14B is a sectional view, taken along line 14A-14A of FIG. 14A, of the urethral-insertion device illustrated in FIG. 14A; and

FIGS. 15A and 15B are longitudinal sectional views of a distal portion of an eleventh embodiment of the urethral-insertion device.

### DETAILED DESCRIPTION

Hereafter, the urethral-insertion device of the present disclosure will be described in detail on the basis of preferred embodiments of the disclosure shown in the attached drawings.

One example of a sling (mesh) 9 to be embedded in a living body will be described below.

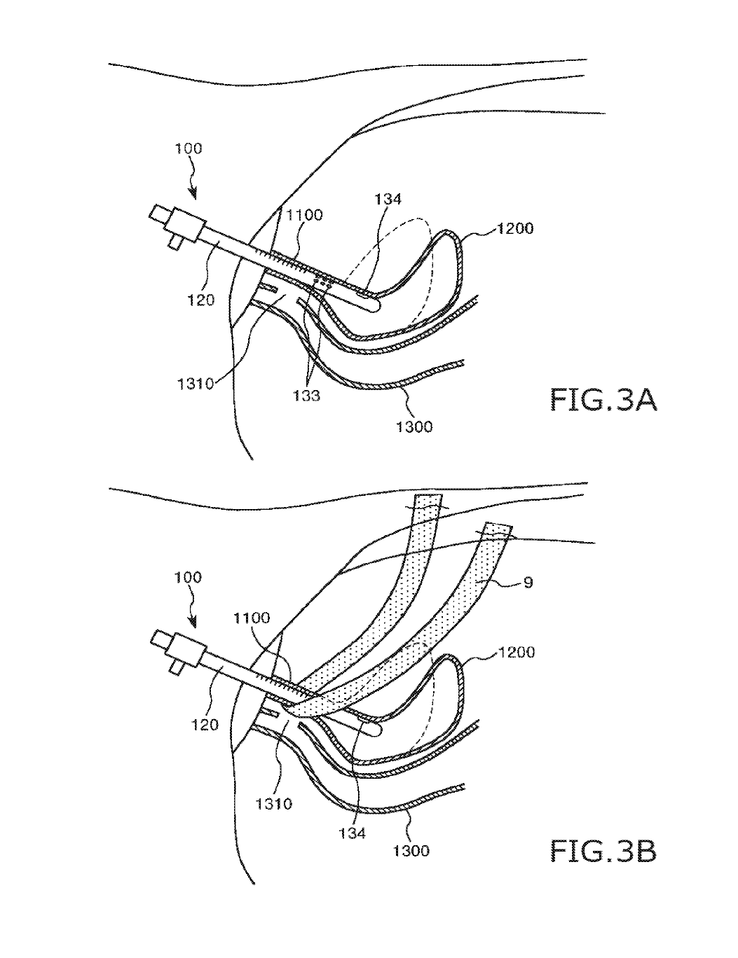

The sling 9 as shown in FIG. 3B is a tool which can be implanted in a living body for treatment of female urinary incontinence. For example, the sling 9 is a tool for supporting a urethra (urethral lumen) 1100 so that when, for example, the urethra 1100 is going to move to the vaginal wall side, the sling 9 restrains such a movement by pulling the urethra 1100 in a direction away from the vaginal wall. As the sling 9 having such a function, for example, an elongate and flexible body can be used.

As illustrated in FIG. 3B, the sling 9 can be net-like in form, and can be belt-like in overall shape. The sling 9 can be composed of a net-like braided or knitted body obtained by intersecting linear (filamentous) members. Examples of the linear (filamentous) members include those which are circular in cross-sectional shape, and those which are flat in cross-sectional shape, namely, which are belt-like or ribbon-like in shape.

The sling 9 configured as above is accommodated in a disinfected wrapping material in an unused state thereof. By this, the sling 9 can be prevented against contamination. The material constituting the sling 9 is not particularly limited; for example, any of various biocompatible resin materials such as polypropylene, polyester, nylon, etc. or fiber or the like thereof can be used. While the sling 9 has been described, the sling 9 is not restricted to the net-shaped one so long as it can exhibit the same or similar effect to the above-mentioned.

One example of a urethral-insertion device configured to be inserted into a urethra 1100 will be described.

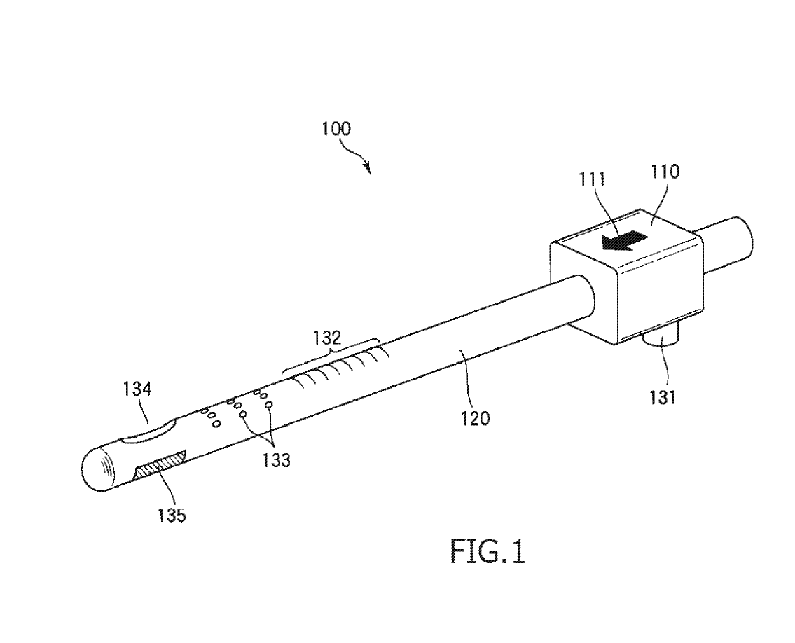

A first embodiment of a urethral-insertion device 100 shown in FIG. 1 includes an elongate shaft (main body part) 120 and a hub 110 supporting the shaft 120. The materials constituting the shaft 120 and the hub 110 are not particularly limited. Examples of the usable materials include various metallic materials such as stainless steel, aluminum or aluminum alloys, titanium or titanium alloys, etc. and various resin materials.

The shaft 120 can include a bladder suction port 134 for attracting a bladder by suction, urethra suction ports 133 disposed on the proximal side of the bladder suction port 134 and attracting a urethra 1100 by suction, an ultrasonic marker 135 disposed opposite to the bladder suction port 134, and a scale 132 disposed on the proximal side of the urethra suction ports 133. The ultrasonic marker 135 can be used to indicate the position of the bladder suction port 134 in a living body. In addition, the scale 132 can be used for measuring the length of insertion of the shaft 120 of the urethral-insertion device 100 inserted in the urethra 1100.

The length of the urethral-insertion device 100 is not specifically restricted, but may be appropriately set according to the length of the patient's urethra 1100, the shape of the patient's bladder 1200, and the like. In view of the fact that the length of a female's urethra 1100 is generally about 30 mm to 50 mm, the length of the urethral-insertion device 100 is preferably, for example, about 50 mm to 300 mm.

It can be preferable, for example, that the urethral-insertion device 100 is in the form of a rigid and straight pipe.

The hub 110 is located on the proximal side of the shaft 120, and can include a mark 111 and a port 131. The mark 111 can be used for indicating the positions of the urethra suction ports 133 and the bladder suction port 134, namely, for indicating the position of the shaft 120 in the circumferential direction. In addition, the port 131 is connected to the urethra suction ports 133 and the bladder suction port 134 through a lumen of the shaft 120. When a suction pump (not shown) is connected to the port 131 and is operated to apply suction, a suction force is generated at each of the urethra suction ports 133 and the bladder suction port 134.

Therefore, when the suction pump is operated in a condition in which the urethral-insertion device 100 is inserted in the urethra 1100 (more specifically, in a condition in which the urethra suction ports 133 are disposed within the urethra 1100 and the bladder suction port 134 is disposed in the bladder 1200), a urethral wall can be fixed by suction onto the urethra suction ports 133, and a bladder wall can be fixed by suction onto the bladder suction port 134. In addition, where a plurality of urethra suction ports 133 with a comparatively small aperture are provided as in the present embodiment, a wide area of the urethral wall can be fixed by suction onto the urethral-insertion device 100 while maintaining a suction force.

When the urethral-insertion device 100 is pushed in toward the inside of a living body (toward the distal side of the shaft 120) in a condition in which the urethral wall is fixed by suction onto the urethra suction ports 133 and the bladder wall is fixed by suction onto the bladder suction port 134, the urethra 1100 and the bladder 1200 are pushed in toward the inside of the body attendant on this, and the bladder 1200 can be shifted to such a position as not to overlap with a puncture route of a puncture needle described later. Further, when the urethral-insertion device 100 is moved toward the vagina side, the position of the bladder 1200 is moved accordingly, whereby the bladder 1200 can be shifted to such a position as not to overlap with the puncture route of the puncture needle. Accordingly, a puncture route for the puncture needle can be relatively secured in a large range, so that puncturing by the puncture needle can be performed relatively accurately and safely.

Note that the number of the urethra suction ports 133 is not particularly limited; for example, only one urethra suction port 133 may be provided. In addition, the layout of the urethra suction ports 133 is not specifically restricted; for example, the urethra suction ports 133 may be formed in only part of the circumferential range of the urethral-insertion device 100 or over the whole range of the circumference.

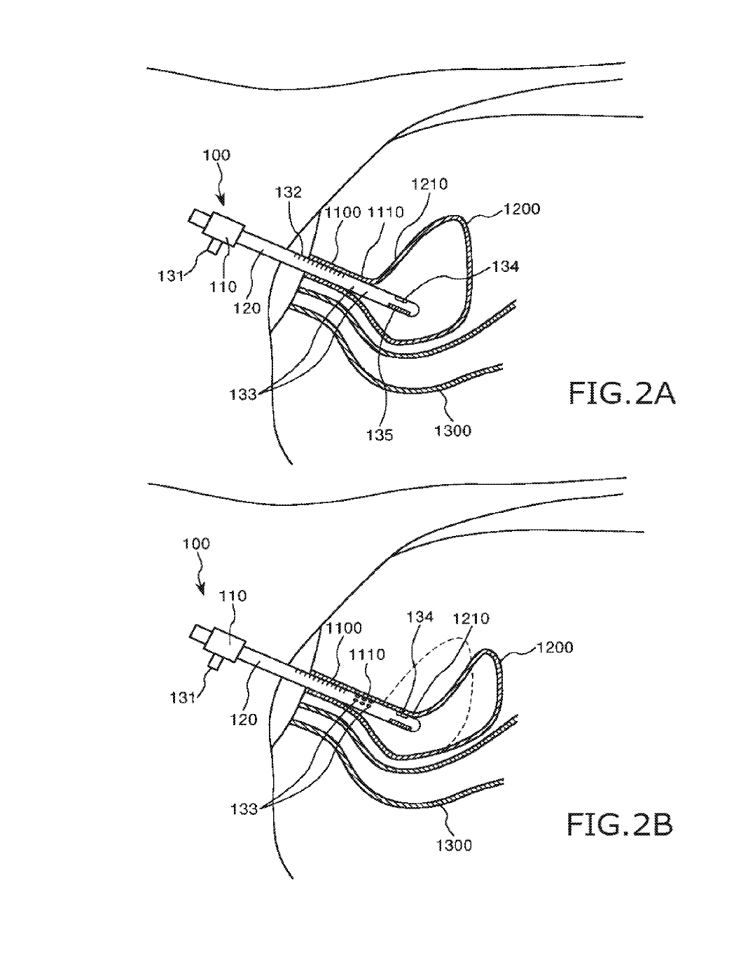

FIGS. 2A, 2B, 3A, and 3B are views illustrating a surgical operation method using the urethral-insertion device 100 (first embodiment) shown in FIG. 1. A surgical operation method for introducing into a living body a sling 9 used in treatment of urinary incontinence by use of the urethral-insertion device 100 can include: a first step of inserting the urethral-insertion device 100 into a urethra 1100; a second step of changing the shape of a bladder 1200 or moving the position thereof in a living body by use of the urethral-insertion device 100 to bring the bladder 1200 away from the puncture route of the puncture needle; and a third step of incising a vagina upper wall of a vagina 1300 and placing the sling 9 indwelling in the living body through the incised part.

Now, the first to third steps will be described in detail below, referring to FIGS. 2A, 2B, 3A, and 3B.

As illustrated in FIG. 2A, in the first step, the urethral-insertion device 100 is first inserted via a urethral orifice of the urethra 1100. In this instance, with the ultrasonic marker 135 and the scale 132 provided at a side surface of the shaft 120 as a reference, the urethra suction ports 133 are located within the urethra 100, and the bladder suction port 134 is located inside the bladder 1200. In addition, a positioning can be conducted so that the mark 111 presents in the outside of the living body is oriented upward, whereby the urethra suction ports 133 and the bladder suction port 134 are oriented upward. In other words, the urethra suction ports 133 are opposed to a urethral upper wall 1110, and the bladder suction port 134 is opposed to a bladder upper wall 1210.

As shown in FIG. 2B, in the second step, suction forces can be generated at the urethra suction ports 133 and the bladder suction port 134 through the port 131, whereby the urethral upper wall 1110 is fixed by suction onto the urethra suction ports 133, and the bladder upper wall 1210 is fixed by suction onto the bladder suction port 134. Then, the urethral-insertion device 100 can be displaced as required, whereby particularly the bladder 1200 is brought away from the puncture route of the puncture needle. With the position of the bladder 1200 changed, retropubic space, which is an extraperitoneal space between the pubic bone and urinary bladder, can be expanded. The retropubic space is the extraperitoneal space between the pubic symphysis and urinary bladder. The retropubic space is also known as “Cave of Retzius” or “Retzius' space,” after Anders Retzius.

In accordance with an exemplary embodiment, for example, with the bladder upper wall 1210 fixed by suction onto the bladder suction port 134, the bladder upper wall 1210 is brought closer to the bladder suction port 134, whereby the shape of the bladder 1200 (the position of the bladder upper wall 1210) is changed. Further, with the urethral-insertion device 100 moved in the direction of the vagina 1300 (in the downward direction) or with the urethral-insertion device 100 moved in the inserting direction (toward the distal side), the position of the bladder 1200 is changed. By thus changing the shape of the bladder 1200 or changing the position of the bladder 1200, the bladder 1200 can be spaced away from the puncture route of the puncture needle, so that mispuncture of the bladder 1200 by the puncture needle can be prevented from occurring.

Particularly, if the urethral-insertion device 100 (the shaft 120) is rigid over the whole length thereof, it can help ensure that when the position or the shape of the bladder 1200 is changed by the urethral-insertion device 100, the urethral-insertion device 100 will not be deformed by a pressure exerted from the biological tissue, and the bladder 1200 can be easily shifted to such a position as not to overlap with the puncture route of the puncture needle.

Note that the broken lines in FIGS. 2B, 3A and 3B indicate the original position of the bladder 1200.

In the third step, first, the vagina upper wall is incised with a surgical knife or the like, to form an incised part 1310, as depicted in FIG. 3A. Next, as shown in FIG. 3B, the puncture needle is introduced into the living body through the incised part 1310 formed in this manner, and an insertion hole for placing the sling 9 indwelling therein is formed. The sling 9 is set indwelling in the living body so as to pass the insertion hole and thereby to support the urethra 1100 from the lower side, whereby it can help prevent urine leakage, which might occur, for example, when an abdominal pressure is exerted. Note that the method of placing the sling 9 indwelling in the living body is not limited to the above-mentioned. For example, a method may be adopted in which the sling 9 is connected to a part of the puncture needle and the sling 9 is introduced into the living body simultaneously with the puncturing by the puncture needle.

Note that the method for placing the sling indwelling in a living body is not restricted to the above-mentioned. The method may include: a first step of inserting the urethral-insertion device into the urethra; a second step of attracting by suction the bladder wall by the bladder suction port provided in the urethral-insertion device; a third step of moving the bladder from its original position with the bladder wall attracted by suction; and a fourth step of placing the sling indwelling in a biological tissue between the urethra and the vagina.

Specifically, the bladder can be spaced away from the puncture route of the puncture needle by moving the bladder from its original position while utilizing the urethral-insertion device.

In addition, for example, a method may be adopted in which the vagina upper wall of the vagina is incised and the sling is set indwelling in the living body through the incision.

In accordance with an exemplary embodiment, the puncture route of the puncture needle may extend from an inguinal region (groin) on one side to an inguinal region on the other side. In this case, the puncture needle is percutaneously inserted from the inguinal region on one side, to form a puncture route, which extends through an obturator foramen on one side, then through a biological tissue between the urethra and the vagina and through an obturator foramen on the other side to reach the inguinal region on the other side. Therefore, an incised part 1310 is not formed in the vagina wall and, accordingly, the invasion to the living body can be minimized.

After the sling 9 is set indwelling, the suction through the urethra suction ports 133 and the bladder suction port 134 is stopped, the urethral-insertion device 100 is withdrawn from the living body, and the incised part 1310 is sutured, to finish the procedure.

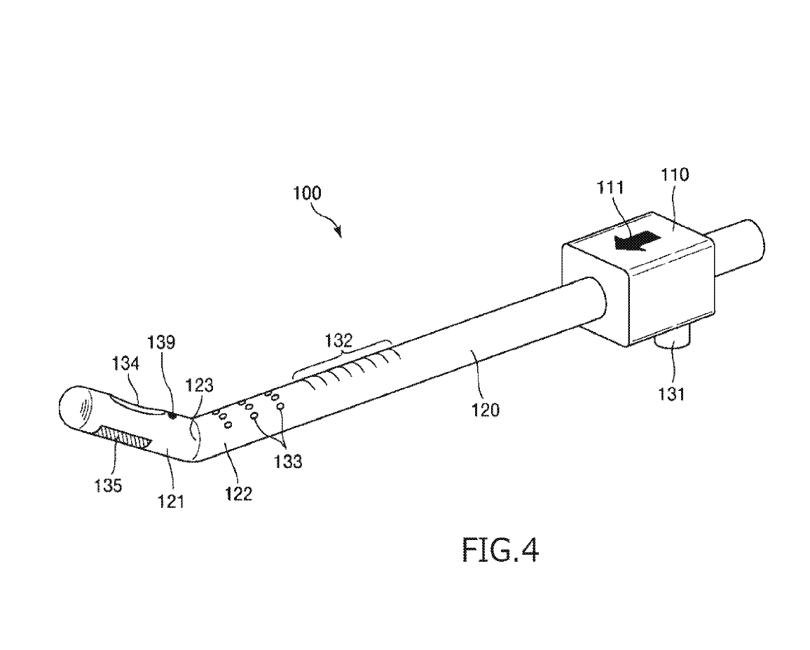

FIG. 4 is a perspective view illustrating a second embodiment of the urethral-insertion device 100.

Referring to this drawing, the second embodiment of the urethral-insertion device 100 will now be described below. The following description focuses on differences of the second embodiment from the first embodiment described above, and the description of the same items as above will be omitted.

This second embodiment is the same as the first embodiment except for a difference in the configuration of the shaft 120.

A urethral-insertion device 100 according to the second embodiment shown in FIG. 4 can include a bladder-placement part 121 provided with a bladder suction port 134, and a urethral-placement part 122 located on a proximal side of the bladder-placement part 121 and provided with urethra suction ports 133. The shaft 120 has a bent part 123 between the bladder suction port 134 and the urethra suction ports 133, and the bladder-placement part 121 is inclined to be oriented upward relative to the urethral-placement part 122, through the bent part 123 therebetween. Therefore, the distance between the upper inner wall of a bladder 1200 and the bladder suction port 134 is shorter than that in the case where the axis of the bladder-placement part 121 and the axis of the urethral-placement part 122 are coincident, and, accordingly, the bladder upper wall 1210 can be easily attracted by suction through the bladder suction port 134.

In addition, the urethral-insertion device 100 in the second embodiment depicted in FIG. 4 has an ultrasonic sensor 139 adapted to transmit and receive ultrasonic waves to and from the bladder-placement part 121, so as to acquire an image of the inside of a biological tissue. The ultrasonic sensor 139 is disposed in the same orientation as the bladder suction port 134 provided in the bladder-placement part 121. Since the ultrasonic sensor 139 transmits and receives ultrasonic waves between itself and the tissue in a body lumen, the biological tissues around a pubic bone and the bladder can be visually confirmed. Therefore, the puncture route of a puncture needle can be confirmed, and puncture by the puncture needle can be performed relatively accurately and safely.

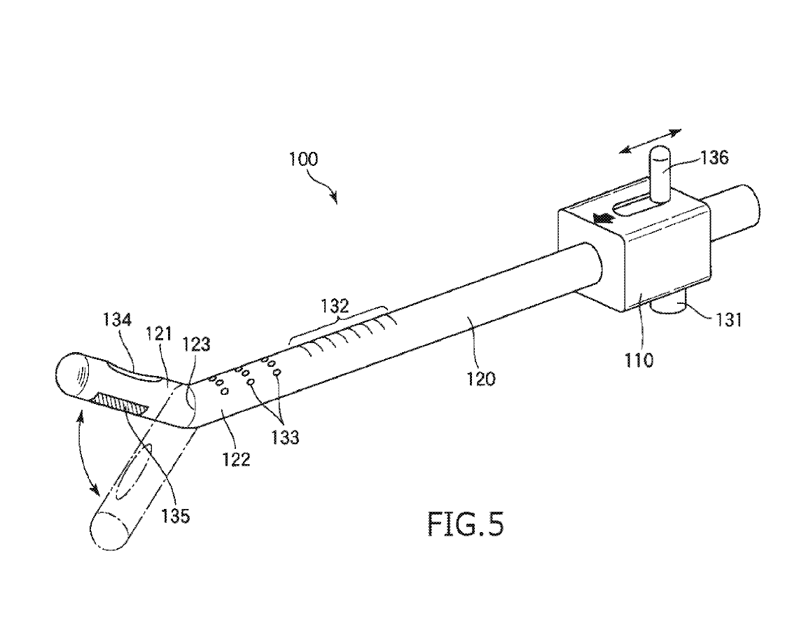

FIG. 5 is a perspective view illustrating a third embodiment of the urethral-insertion device 100.

Referring to this drawing, the third embodiment of the urethral-insertion device 100 will now be described below. The following description focuses on differences of the third embodiment from the embodiments described above, and the description of the same items as above will be omitted.

This third embodiment is the same as the second embodiment above except for a difference in the configuration of the shaft 120.

A urethral-insertion device 100 according to the third embodiment shown in FIG. 5 is so configured that the axis of a bladder-placement part 121 can be displaced in relation to the axis of a urethral-placement part 122. Specifically, as depicted in FIG. 5, the bladder-placement part 121 is configured to be bent upward and downward in relation to the urethral-placement part 122. Note that a lever 136 is connected to a bent part 123 through a lumen of the shaft 120, and, when the lever 136 is operated in forward/backward direction, the bladder-placement part 121 of the shaft 120 can be bent in upward/downward direction through the bent part 123.

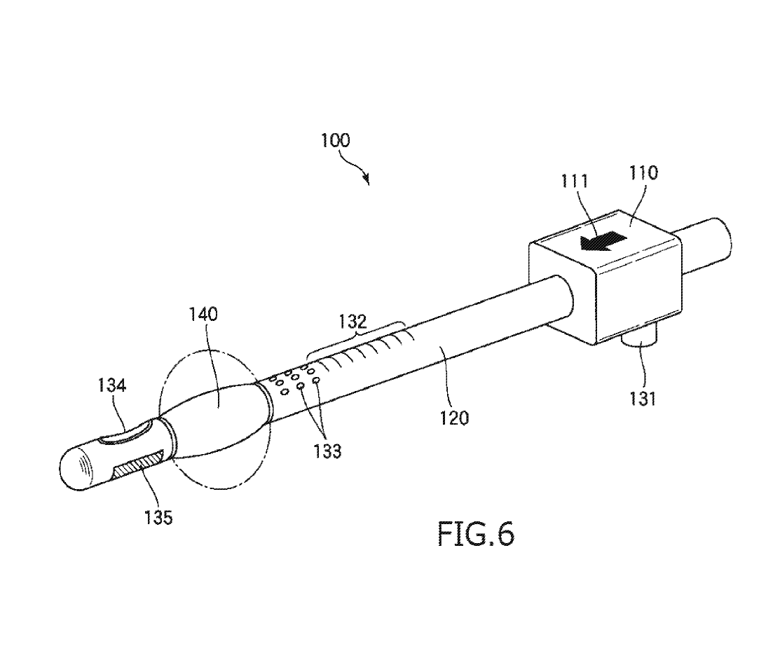

FIG. 6 is a perspective view of a fourth embodiment of the urethral-insertion device 100.

Referring to this drawing, the fourth embodiment of the urethral-insertion device 100 will now be described below. The following description focuses on differences of the fourth embodiment from the embodiments described above, and the description of the same items as above will be omitted.

The fourth embodiment is the same as the first embodiment above except for a difference in the configuration of the shaft 120.

A urethral-insertion device 100 according to the fourth embodiment shown in FIG. 6 has, between urethra suction ports 133 and a bladder suction port 134, a balloon (contact part) 140 which is an expansion body having flexibility and capable of being expanded and contracted. The balloon 140 is so disposed as to be located inside a bladder 1200 when the urethral-insertion device 100 is inserted in a urethra 1100.

The balloon 140 is connected to a balloon port (not shown) on a proximal side of the urethral-insertion device 100 through a lumen of the shaft 120.

A balloon expanding device such as a syringe can be connected to the balloon port. When a working fluid (a liquid such as physiological salt solution or a gas or the like) is supplied from the balloon expanding device into the balloon 140, the balloon 140 is expanded. Conversely, when the working fluid is drawn out of the balloon 140 by the balloon expanding device, the balloon 140 is contracted. With the balloon 140 expanded after inserted into a bladder 1200 to be thereby pressed against a bladder neck, the urethral-insertion device 100 can be disposed into an appropriate position.

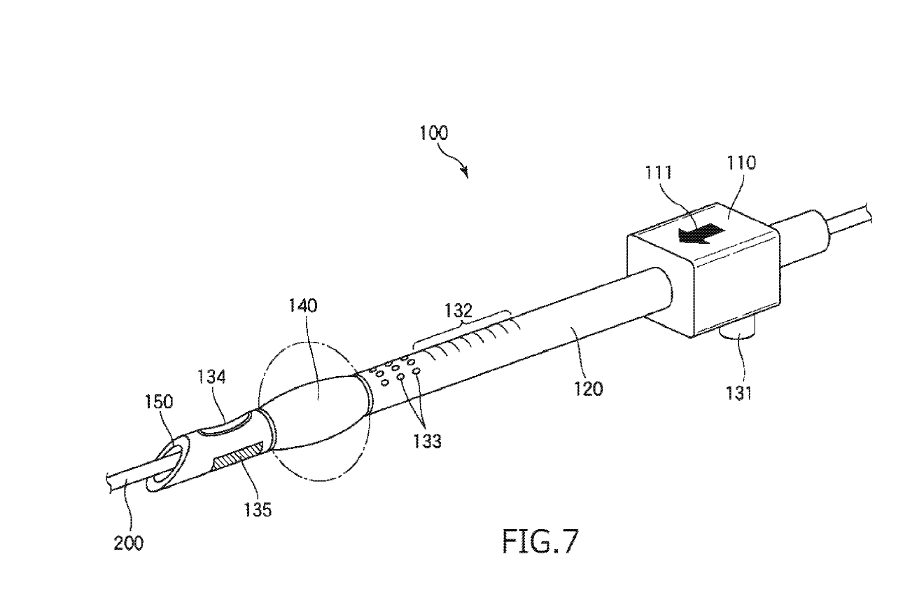

FIG. 7 is a perspective view illustrating a fifth embodiment of the urethral-insertion device 100.

Referring to this drawing, the fifth embodiment of the urethral-insertion device 100 will now be described below. The following description focuses on differences of the fifth embodiment from the embodiments described above, and the description of the same items as above will be omitted.

This fifth embodiment is the same as the first embodiment above except for a difference in the configuration of the shaft 120.

A urethral-insertion device 100 according to the fifth embodiment shown in FIG. 7 is provided with a through-hole 150 penetrating the shaft 120 along the axial direction of the shaft 120, and a stylet 200 is slidable through the through-hole 150. After the urethral-insertion device 100 is inserted into a urethra 1100, the stylet 200 is inserted into the through-hole 150, and the stylet 200 is protruded from a distal opening of the through-hole 150. In this embodiment, the shaft 120 of the urethral-insertion device 100 is lower than the stylet 200 in rigidity. Therefore, the shaft 120 can be deformed in shape according to the shape of the stylet 200 inserted in the through-hole 150. For instance, when a stylet 200, which is partly bent is used, the shape of the shaft 120 is deformed following the shape of the stylet 200. Therefore, with the shaft 120 so bent as to approach a bladder upper wall 1210, the distance between a bladder suction port 134 and the bladder upper wall 1210 can be shortened, so that the bladder upper wall 1210 can be more easily attracted and fixed by suction.

In addition, after the urethral-insertion device 100 is inserted in the urethra 1100, the stylet 200 is inserted into the through-hole 150, and the stylet 200 is protruded from the distal opening of the through-hole 150. Next, a depth wall of a bladder 1200 may be pushed in by the stylet 200, thereby moving the position of the bladder 1200. By this, the position of the bladder 1200 can be spaced away from a puncture route of a puncture needle. Besides, with the depth wall of the bladder 1200 pushed, a bladder upper wall 1210 is moved toward the vagina side. Therefore, the distance between the bladder suction port 134 and the bladder upper wall 1210 is shortened, so that the bladder upper wall 1210 can be more easily attracted and fixed by suction.

Note that the material constituting the stylet 200 is not particularly limited. Examples of the usable material include various metallic materials such as stainless steel, aluminum or aluminum alloys, titanium or titanium alloys, etc. and various resin materials.

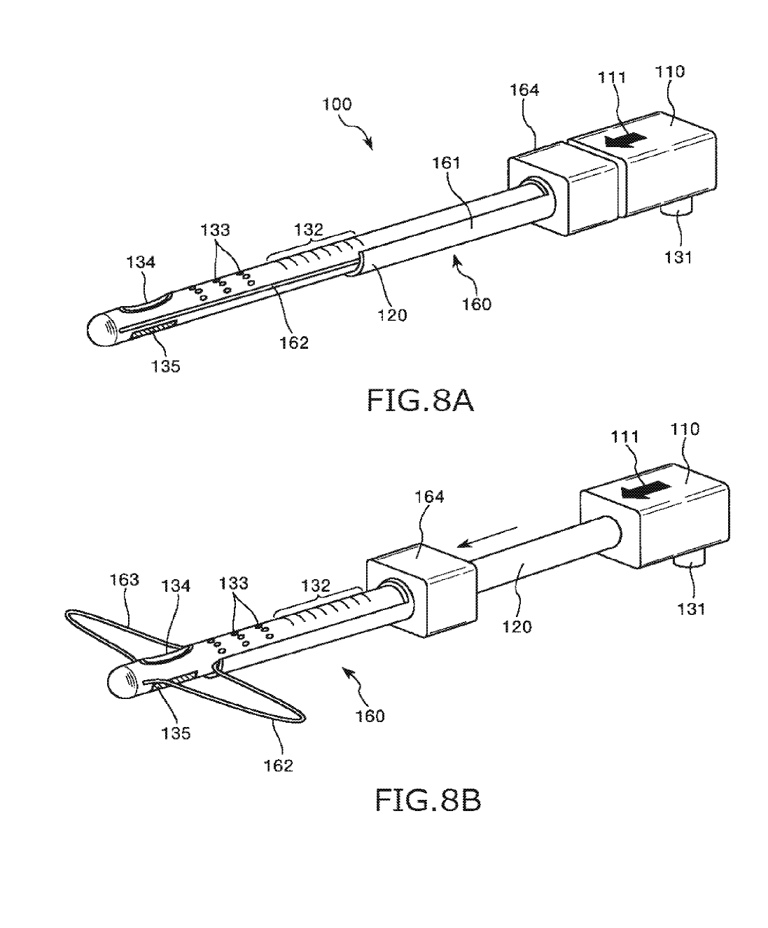

FIGS. 8A and 8B are perspective views illustrating a sixth embodiment of the urethral-insertion device 100.

Referring to these drawings, the sixth embodiment of the urethral-insertion device 100 will now be described below. The following description focuses on differences of the sixth embodiment from the embodiments described above, and the description of the same items as above will be omitted.

The sixth embodiment is the same as the first embodiment except for having a pushing part 160.

A urethral-insertion device 100 according to the sixth embodiment shown in FIG. 8A has the pushing part 160 adapted to push a bladder 1200 from inside to change the shape of the bladder 1200. The pushing part 160 can include a slide pipe 161 slidable relative to a shaft 120, an operation part 164 capable of an operation to slide the slide pipe 161, and extension wires 162 and 163 which are deformed by a sliding operation of the operation part 164.

In accordance with an exemplary embodiment, the slide pipe 161 is pipe-like in shape, is disposed to cover an outer periphery of the shaft 120, and is slidable forward and backward on the outer peripheral surface of the shaft 120 along the outer peripheral surface of the shaft 120. Note that the slide pipe 161 can be provided with a slit for preventing the slide pipe 161 from closing urethra suction ports 133 when the slide pipe 161 is slid. The operation part 164 is located on a distal side of a hub 110, and is connected to the slide pipe 161. Therefore, the slide pipe 161 is slid forward and backward on the outer peripheral surface of the shaft 120 by the sliding operation of the operation part 164. In addition, the hub 110 may have a function as a stopper for stopping the sliding operation of the operation part 164.

In accordance with an exemplary embodiment, the extension wires 162 and 163 are each composed of wire, and are disposed with distal portions of the extension wires 162 and 163 fixed to the distal side of the shaft 120 and with proximal portions of the extension wires 162 and 163 fixed to the distal side of the slide pipe 161. Besides, the extension wires 162 and 163 are disposed opposite to each other, with the shaft 120 between the extension wires 162, 163. With the slide pipe 161 slid along the shaft 120 by a sliding operation of the operation part 164, the distance between the distal end of the slide pipe 161 and the distal end of the shaft 120 is changed. By this, the distance between distal end portions and proximal end portions of the extension wires 162 and 163 can be changed, so that the shapes of the extension wires 162 and 163 are deformed.

Specifically, when the operation part 164 is operated for sliding in an inserting direction, starting from a condition in which the extension wires 162 and 163 are rectilinear in shape as shown in FIG. 8A, the slide pipe 161 is moved in the inserting direction. By the movement of the slide pipe 161, the distance between the distal end portions and the proximal end portions of the extension wires 162 and 163 is shortened as shown in FIG. 8B, so that the extension wires 162 and 163 are deformed to protrude in radially outward directions of the shaft 120.

After the extension wires 162 and 163 are inserted into a bladder 1200, left and right walls of the bladder 1200 can be pushed by deforming the extension wires 162 and 163 to protrude in the radially outward directions of the shaft 120. With the left and right walls of the bladder 1200 thus pushed, the bladder 1200 is broadened in the lateral direction, and a bladder upper wall 1210 is moved in a downward direction (in a direction toward a vagina) accordingly, so that the distance between the bladder upper wall 1210 and a bladder suction port 134 is shortened. Consequently, the bladder upper wall 1210 can be easily attracted by suction.

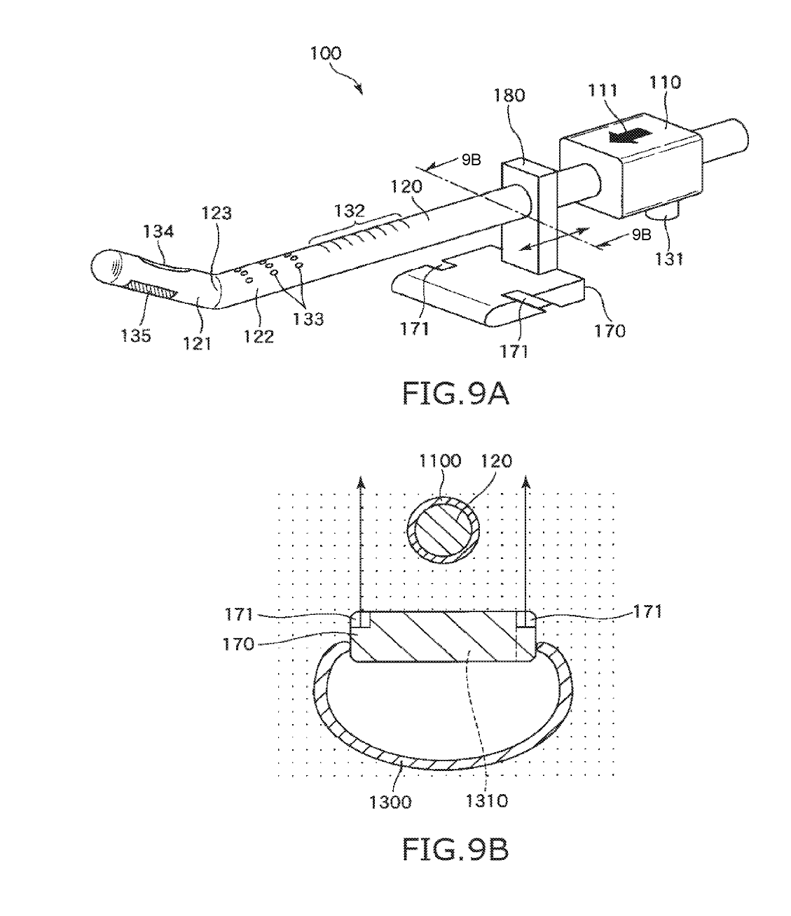

FIG. 9A is a perspective view illustrating a seventh embodiment of the urethral-insertion device 100, and FIG. 9B is a sectional view of the urethral-insertion device 100 taken along line 9B-9B of FIG. 9A.

Referring to these drawings, the seventh embodiment of the urethral-insertion device 100 will now be described below. The following description focuses on differences of the seventh embodiment from the embodiments described above, and the description of the same items as above will be omitted.

This seventh embodiment is the same as the second embodiment above except for having a vaginal-insertion member 170 connected to the urethral-insertion device 100.

A urethral-insertion device 100 shown in FIG. 9A has the vaginal-insertion member 170. The vaginal-insertion member 170 is configured to be slidable forward and backward in relation to a shaft 120 through a connection part 180.

The materials constituting the vaginal-insertion member 170 and the connection part 180 are not particularly limited. Various metallic materials such as stainless steel, aluminum or aluminum alloys, titanium or titanium alloys, etc. and various resin materials can be used for the vaginal-insertion member 170 and the connection part 180, like for the shaft 120.

The vaginal-insertion member 170 has guiding grooves 171 for guiding a puncture needle for puncturing a biological tissue. The guiding grooves 171 are present on both sides (left and right sides) of the vaginal-insertion member 170. In top plan view in FIG. 9A, the shaft 120 is located between upper-side openings of the two guiding grooves 171 so as not to overlap with the upper-side openings. In addition, the guiding grooves 171 are provided in an inclined form such that the upper-side openings thereof are located on the more distal side than the lower-side openings thereof. Since the urethral-insertion device 100 has the vaginal-insertion member 170, the puncture needle can puncture a biological tissue while avoiding a urethra 1100.

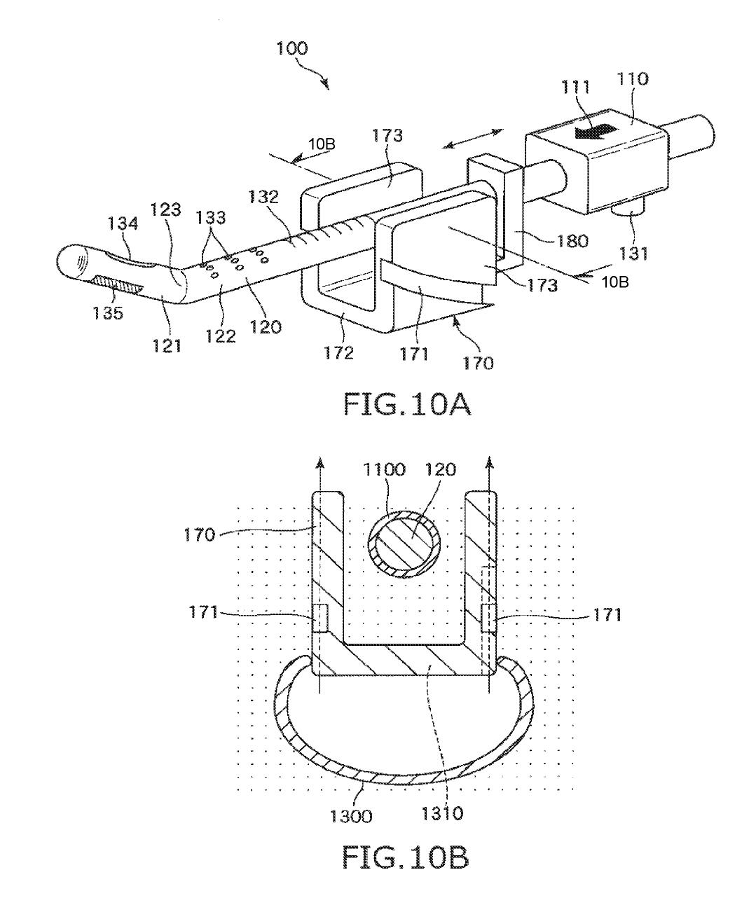

FIG. 10A is a perspective view illustrating an eighth embodiment of the urethral-insertion device 100, and FIG. 10B is a sectional view of the urethral-insertion device 100 taken along line 10B-10B of FIG. 10A.

Referring to these drawings, the eighth embodiment of the urethral-insertion device 100 will now be described below. The following description focuses on differences of the eighth embodiment from the embodiments described above, and the description of the same items as above will be omitted.

This eighth embodiment is the same as the seventh embodiment except for a difference in the configuration of the vaginal-insertion member 170 connected to the urethral-insertion device 100.

As shown in FIG. 10A, the vaginal-insertion member 170 can include a support part 172, a pair of side wall parts 173, and guiding grooves 171 for guiding a puncture needle for puncturing a biological tissue. The side wall parts 173 are provided on both sides of the support part 172, and are disposed opposite to each other, with a shaft 120 between the side walls 173.

The guiding grooves 171 are each present in the side wall parts 173, and are each present in an inclined form to extend gradually upward from a proximal-side opening thereof located on the proximal side of the side wall part 173 toward a distal-side opening thereof located on the distal side of the side wall part 173. The support part 172 and the pair of side wall parts 173 of the vaginal-insertion member 170 have a roughly U-shaped overall sectional form such as to surround the shaft 120. With the urethral-insertion device 100 thus provided with the vaginal-insertion member 170, the puncture needle can puncture a biological tissue while avoiding a urethra 1100.

Note that the vaginal-insertion member 170 may be so configured that it can be attached to and detached from the shaft 120 through a connection part 180. In this case, the vaginal-insertion member 170 can be attached to the connection part 180 after insertion of the shaft 120 into the urethra 1100. Accordingly, the urethral-insertion device 100 can be disposed at an appropriate position relatively easily.

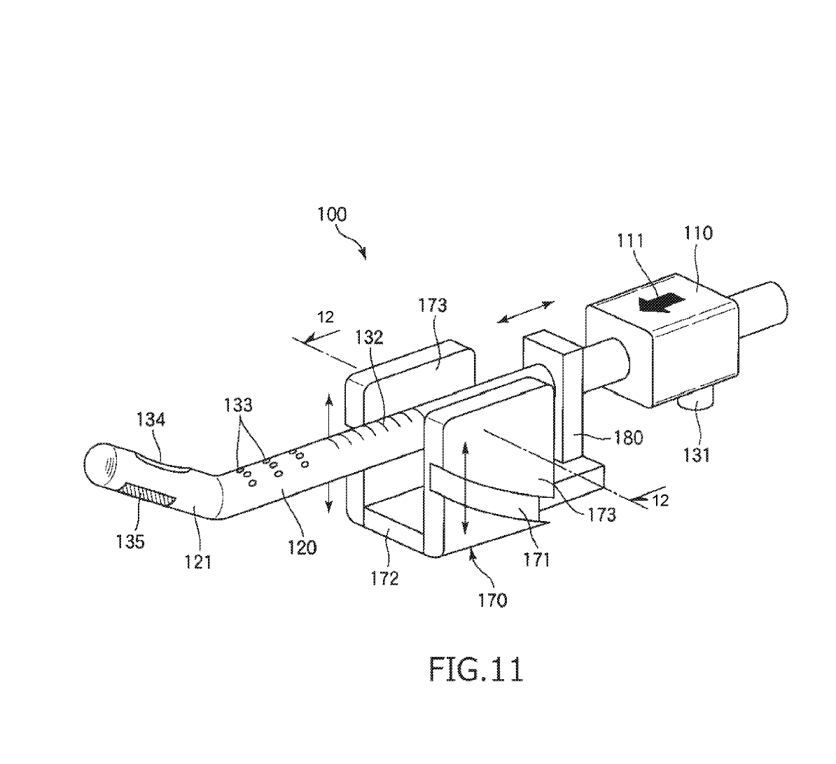

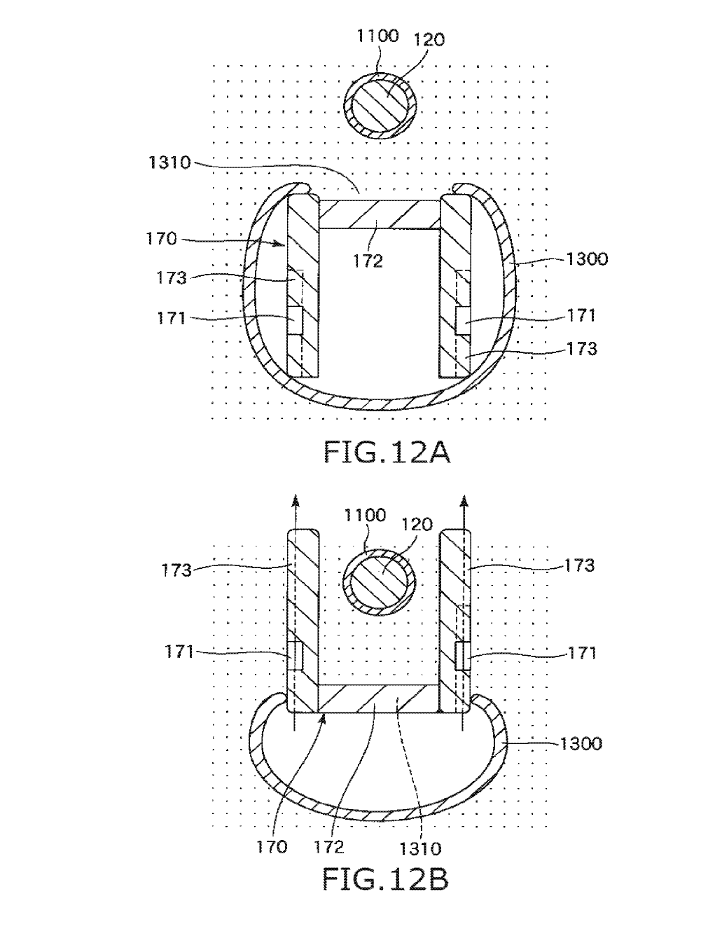

FIG. 11 is a perspective view illustrating a ninth embodiment of the urethral-insertion device 100, and FIGS. 12A and 12B are sectional views of the urethral-insertion device 100 taken along line 12-12 of FIG. 11.

Referring to these drawings, the ninth embodiment of the urethral-insertion device 100 will now be described below. The following description focuses on differences of the ninth embodiment from the embodiments described above, and the description of the same items as above will be omitted.

This ninth embodiment is the same as the eighth embodiment except for a difference in the configuration of the vaginal-insertion member 170 connected to the urethral-insertion device 100.

As shown in FIG. 11, the vaginal-insertion member 170 includes a support part 172, a side wall parts 173 disposed on both sides of the support part 172, and guiding grooves 171 formed in the side wall parts 173. As depicted in FIGS. 12A and 12B, the side wall parts 173 can be configured to be vertically slidable relative to the support part 172. Therefore, the side wall parts 173 can be inserted into a living body through an incised part 1310 after the insertion of the vaginal-insertion member 170 into a vagina 1300. Accordingly, the vaginal-insertion member 170 can be easily inserted into the vagina 1300.

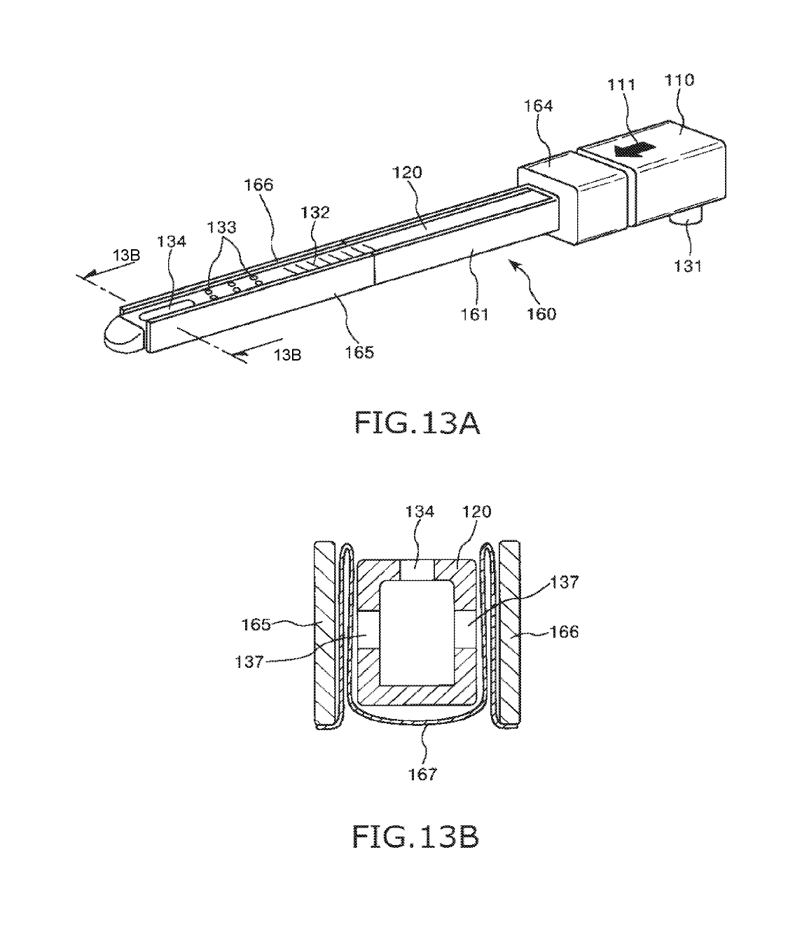

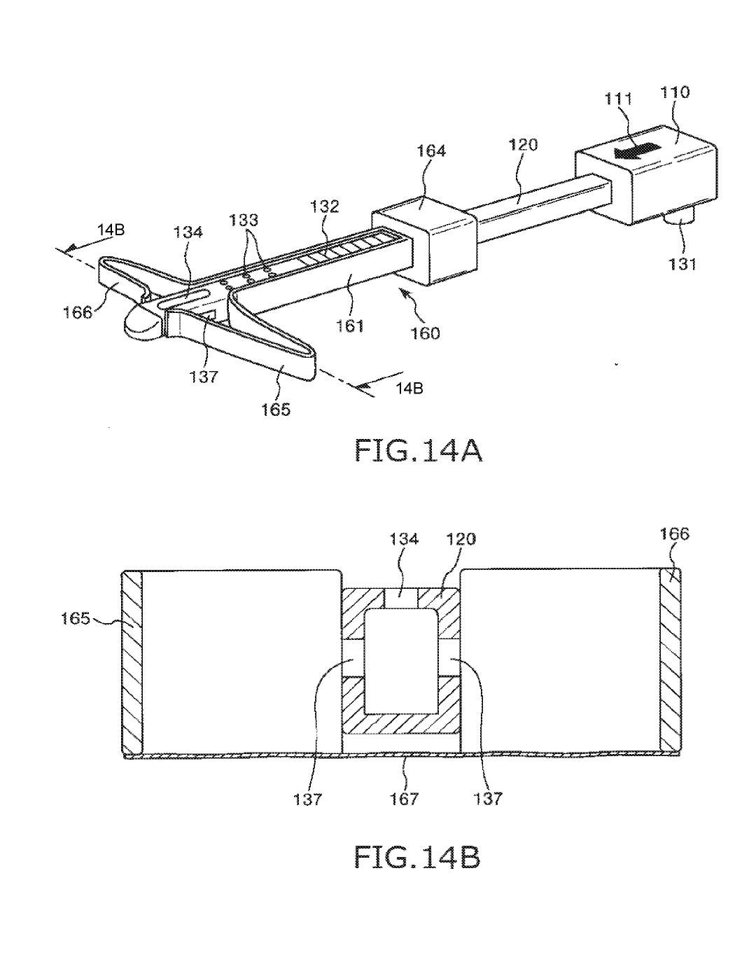

FIGS. 13A and 14A are perspective views illustrating a tenth embodiment of the urethral-insertion device 100, FIG. 13B is a sectional view of the urethral-insertion device 100 taken along line 13B-13B of FIG. 13A, and FIG. 14B is a sectional view of the urethral-insertion device 100 taken along line 14B-14B of FIG. 14A.

Referring to these drawings, the tenth embodiment of the urethral-insertion device 100 will now be described below. The following description focuses on differences of the tenth embodiment from the embodiments described above, and the description of the same items as above will be omitted.

This tenth embodiment is the same as the sixth embodiment above except for a difference in the configuration of the pushing part 160 provided in the urethral-insertion device 100 and a difference in the position of a suction surface of the bladder suction port 134.

In accordance with an exemplary embodiment, the pushing part 160 shown in FIG. 13A can include: a slide pipe 161 slidable relative to a shaft 120; an operation part 164 capable of an operation to slide the slide pipe 161; a pair of extension plates 165 and 166 deformed by a sliding operation of the operation part 164; and a membrane part 167 connecting the extension plates 165 and 166.

The extension plates 165 and 166 are each composed of a plate-shaped body, and are disposed with distal end portions of the extension plates 165 and 166 fixed to a distal end portion of the shaft 120 and with proximal end portions of the extension plates 165 and 166 fixed to a distal end portion of the slide pipe 161. In addition, the extension plates 165 and 166 are disposed opposite to each other, with the shaft 120 between the extension plates 165, 166. With the slide pipe 161 slid along the shaft 120 by the sliding operation of the operation part 164, the distance between the distal end of the slide pipe 161 and the distal end of the shaft 120 is changed. By this, the distance between the distal end portions and the proximal end portions of the extension plates 165 and 166 is changed, so that the shape of the extension plates 165 and 166 is deformed.

FIG. 13B depicts a condition before the extension plates 165 and 166 are expanded in the radial direction by the sliding operation of the slide pipe 161. As shown in FIG. 13B, the membrane part 167 is disposed in the state of being connected to lower surfaces of the extension plates 165 and 166. Besides, in the condition before extension of the extension plates 165 and 166, the membrane part 167 is disposed in the state of being folded between the shaft 120 and the extension plates 165 and 166.

FIG. 14A is a perspective view illustrating a condition after the extension plates 165 and 166 are extended, and FIG. 14B is a sectional view taken along line 14B-14B of FIG. 14A.

As shown in FIG. 14A, in the extended state, a space surrounded by the extension plates 165 and 166 and the membrane part 167 can be formed, and a suction force generated from the bladder suction port 134 can be distributed to the whole upper region of the space. Therefore, a bladder upper wall 1210 can be attracted and fixed by suction over a broad range.

In addition, as shown in FIG. 14B, in this embodiment, not only the bladder suction port 134 is provided in the upper surface of the shaft 120 but also a plurality of bladder suction ports 134 are provided in side surfaces of the shaft 120. Therefore, even if the bladder suction port 134 in the upper surface is closed with the bladder upper wall 1210 attracted thereto by suction, suction forces are generated from the bladder suction ports 134 in the side surfaces, so that the suction force generated at the upper side of the space surrounded by the extension plates 165 and 166 and the membrane part 167 can be maintained.

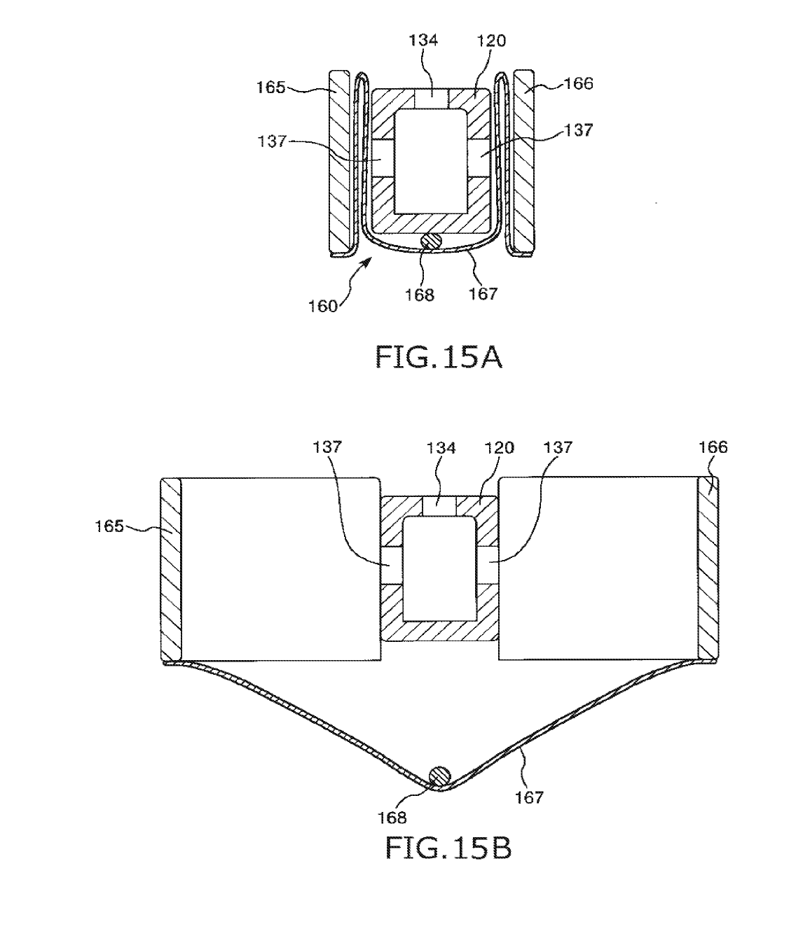

FIGS. 15A and 15B are longitudinal sectional views of a distal portion of a shaft, illustrating an eleventh embodiment of the urethral-insertion device 100, wherein FIG. 15A depicts a condition before extension of extension plates 165 and 166, and FIG. 15B depicts a condition after extension of the extension plates 165 and 166.

Referring to these drawings, the eleventh embodiment of the urethral-insertion device 100 will now be described below. The following description focuses on differences of the eleventh embodiment from the embodiments described above, and the description of the same items as above will be omitted.

This eleventh embodiment is the same as the tenth embodiment except for a difference in the configuration of the pushing part 160 provided in the urethral-insertion device 100.

The pushing part 160 shown in FIG. 15A has an extension wire 168 which can be extended downward.

The extension wire 168 is composed of wire, and is disposed with a distal end portion of the extension wire 168 fixed to a distal end portion of a shaft 120 and with a proximal end portion of the extension wire 168 fixed to a distal end portion of a slide pipe 161. With the slide pipe 161 slid along the shaft 120 by an operation for sliding of an operation part 164, the distance between the distal end of the slide pipe 161 and the distal end of the shaft 120 is changed. By this, the distance between the distal end portion and the proximal end portion of the extension wire 168 is changed, so that the extension wire 168 is deformed to protrude in the radially outward direction.

In the above embodiments, description has been made of the case where the urethral-insertion device is applied to a device for use in embedding in a living body a sling embeddable for treatment of female urinary incontinence. The use of the urethral-insertion device, however, is not limited to the described one.

For example, the target of the application of the present disclosure includes excretory disorders attendant on the weakening of the pelvic floor muscle group (urinary urgency, frequent urination, urinary incontinence, fecal incontinence, urinary retention, strangury or the like), and pelvic floor disorders including pelvic organ prolapse, vesicovaginal fistula, urethrovaginal fistula, pelvic pain or the like. In the pelvic organ prolapse, there are included disorders of cystocele, enterocele, rectocele, hysterocele and the like. Alternatively, there are included such disorders as anterior vaginal prolapse, posterior vaginal prolapse, vaginal apical prolapse, vaginal vault prolapse and the like in which the naming method thereof is based on the prolapsed vaginal-wall part.

Also, overactive tissues include bladder, vagina, uterus, bowel and the like. Less active tissues include bones, muscles, fascias, ligaments and the like. In particular, in the case of pelvic floor disorders, the less active tissues include an obturator fascia, a coccygeus fascia, a cardinal ligament, an uterosacral ligament, a sacrospinous ligament and the like.

For the procedure for interlocking an overactive tissue in the pelvic floor disorder with the less active tissue, there are included retropubic sling surgery, transobturator sling surgery, transobturator tape (TOT), tension-free vaginal mesh (TVM), uterosacral ligament suspension (USLS), sacrospinous ligament fixation (SSLF), iliococcygeus fascia fixation surgery, coccygeus fascia fixation surgery, and the like.

The detailed description above describes a urethral-insertion device. The disclosure is not limited, however, to the precise embodiments and variations described. Various changes, modifications and equivalents can be effected by one skilled in the art without departing from the spirit and scope of the disclosure as defined in the accompanying claims. It is expressly intended that all such changes, modifications and equivalents which fall within the scope of the claims are embraced by the claims.

What is claimed is:

1. A urethral-insertion device configured to be inserted into a urethra, the urethral-insertion device comprising:
an elongated main body;
a bladder suction port adapted to attract a bladder by suction, the bladder suction port being located on the main body; and
a urethra suction port adapted to attract a urethra wall by suction, the urethra suction port being located on a distal side of the bladder suction port.

2. The urethral-insertion device according to claim 1, further comprising:
a bladder-placement part provided with the bladder suction port;
a urethral-placement part provided with the urethra suction port; and
a bent part located between the bladder-placement part and the urethral-placement part,
wherein an axis of the bladder-placement part is arranged to be inclined against an axis of the urethral-placement part.

3. The urethral-insertion device according to claim 2, wherein the axis of the bladder-placement part is movable with respect to the axis of the urethral-placement part.

4. The urethral-insertion device according to claim 1, further comprising:
a stylet configured to be inserted into a lumen of the main body,
wherein a shape of the main body is configured to be changeable to follow a shape of the stylet.

5. The urethral-insertion device according to claim 1, further comprising:
a pushing part adapted to push an inner wall of the bladder.

6. The urethral-insertion device according to claim 1, further comprising:
a vaginal-insertion member configured to be inserted into a vagina,
wherein the vaginal-insertion member includes a guiding groove for guiding a puncture needle for puncturing a living body tissue.

7. The urethral-insertion device according to claim 6, wherein the vaginal-insertion member includes a support part and a pair of side wall parts connected to the support part, and the pair of side wall parts facing each other.

8. The urethral-insertion device according to claim 1, further comprising:
an ultrasonic marker on a distal portion of the main body, the ultrasonic marker being configured to transmit and receive ultrasonic waves,
wherein the bladder suction port and the urethra suction port are arranged on one side of the main body, and the ultrasonic marker is on an opposite side of the main body.

9. The urethral-insertion device according to claim 1, further comprising:
an inflatable and deflatable balloon located between the bladder suction port and the urethra suction port.

10. The urethral-insertion device according to claim 1, further comprising:
a pair of extension wires on a distal portion of the main body.

11. The urethral-insertion device according to claim 1, further comprising:
a pair of extension plates on a distal portion of the main body.

12. A method of placing a sling for use in treatment of urinary incontinence indwelling into a living body, the method comprising:
inserting a urethral-insertion device into a urethra;
attracting by suction an inner wall of a bladder by a bladder suction port of the urethral-insertion device;
moving the bladder from an original position of the bladder; and
placing the sling indwelling into the living body between a vagina and the urethra.

13. The method according to claim 12, further comprising:
diverting the bladder from a puncture route of a puncture needle by moving the bladder from the original position.

14. The method according to claim 12, further comprising:
incising a vaginal wall; and
placing the sling indwelling into the living body through an incision of the vaginal wall.

15. The method according to claim 12, wherein the puncture route of the puncture needle extends from an inguinal region on one side to an inguinal region on the other side.

16. The method according to claim 12, wherein the urethral-insertion device includes
an elongated main body,
a bladder suction port adapted to attract a bladder by suction, the bladder suction port being located on the main body; and
a urethra suction port adapted to attract a urethra wall by suction, the urethra suction port being located on a distal side of the bladder suction port.

17. The method according to claim 16,
wherein the urethral-insertion device further includes a bladder-placement part provided with the bladder suction port, an urethral-placement part provided with the urethral suction port, and a bent part located between the bladder-placement part and the urethral-placement part; and
an axis of the bladder placement part is arranged to be inclined against an axis of the urethra placement part.

18. The method according to claim 17, wherein the axis of the bladder placement part is movable with respect to the axis of the urethra placement part.

19. The method according to claim 16, further comprising:
inserting a stylet into a lumen of the main body; and
changing a shape of the main body to follow a shape of the stylet.

20. The method according to claim 12, further comprising:
inserting a vaginal-insertion member into a vagina, and wherein the vaginal-insertion member includes a guiding groove for guiding a puncture needle for puncturing a living body tissue.
